(19) United States
(12) Patent Application Publication
(10) Pub. No.: US 2021/0325389 A1
(43) Pub. Date: Oct. 21, 2021

(54) System and Method for Obtaining Post-Infection Clearance

(71) Applicant: Medic, Inc., Provo, UT (US)

(72) Inventors: David R. Hall, Provo, UT (US); K. Jeffrey Campbell, Spanish Fork, UT (US); Anthony E. Pullen, Tucson, AZ (US); Jeffery Duncan, Tucson, AZ (US)

(73) Assignee: Medic, Inc., Provo, UT (US)

(21) Appl. No.: 17/231,937

(22) Filed: Apr. 15, 2021

Related U.S. Application Data
(63) Continuation-in-part of application No. 17/226,001, filed on Apr. 8, 2021.
(60) Provisional application No. 63/010,604, filed on Apr. 15, 2020.

(51) Int. Cl. G01N 33/569; G01N 33/53

(52) U.S. Cl. CPC G01N 33/56983; G01N 33/5302; G01N 2800/26; G01N 2469/20; G01N 2333/165

(57) ABSTRACT

A system for obtaining clearance comprises a mechanism to identify the user and generate an identification signal; an analytical toilet comprising a bowl to receive excreta from the user; an analyzer configured to analyze the sample to detect one or more antibodies in a sample of the excreta or sputum and to generate an analysis signal; a processor configured to receive the identification signal and the analysis signal; determine therefrom the status of the user with respect to the one or more antibodies; and transmit a report to an authority, the report comprising the identity and the status of the user with respect to the antibodies detected. A method for obtaining clearance comprises linking a user to an analytical toilet; receiving a sample of excreta from the user in the analytical toilet; analyzing the sample of excreta in an analytical toilet to detect one or more antibodies; processing results from the analytical toilet and determining the status of the user with respect to the antibodies detected; submitting the identity and the status of the user with respect to the antibodies detected to an authority.

### CROSS-REFERENCE TO RELATED APPLICATIONS

This application is a continuation-in-part of U.S. patent application Ser. No. 17/226,001 titled “System and Method for Obtaining Clearance” filed 8 Apr. 2021. This application also claims priority to U.S. Provisional Patent Application No. 63/010,604 titled “Method for Obtaining Post-Infection Clearance” filed on 15 Apr. 2020, which is incorporated herein by reference in its entirety.

### TECHNICAL FIELD

The present disclosure relates to a method to obtain clearance, such as a medical clearance following recovery from an infection disease.

### BACKGROUND

The ability to track an individual's health and wellness is currently limited due to the lack of available data related to personal health. Many diagnostic tools are based on examination and testing of bodily fluids, such as blood, saliva, and excreta, but the high cost of frequent doctor's visits and/or scans make these options available only on a very limited and infrequent basis. Thus, they are not widely available to people interested in tracking their own personal wellbeing.

### SUMMARY

In a first embodiment, the disclosure provides a system for obtaining clearance for a user comprising a mechanism to identify the user and generate an identification signal; an analytical toilet comprising a bowl to receive excreta and sputum from the user; an analyzer configured to analyze the sample to detect one or more antibodies in a sample of the excreta or sputum and to generate an analysis signal; a processor configured to receive the identification signal and the analysis signal; determine therefrom the status of the user with respect to the one or more antibodies; and transmit a report to an authority, the report comprising the identity of the user and the status of the user with respect to the antibodies detected.

In a second embodiment, the disclosure provides a method for obtaining travel clearance for a user comprising linking a user to an analytical toilet; receiving a sample of excreta or sputum from the user in the analytical toilet; analyzing the sample of excreta or sputum in an analytical toilet to detect one or more antibodies; processing results from the analytical toilet and determining the status of the user with respect to the antibodies detected; submitting the identity of the user and the status of the user with respect to the antibodies detected to an authority for the purpose of gaining clearance from the authority.

In a third embodiment, the disclosure provides a method for obtaining clearance for a user comprising providing an analytical toilet system with the capability to identify the user and to detect one or more antibodies by analyzing a sample of excreta or sputum from the user; identifying the user; receiving a sample of excreta or sputum from the user in the analytical toilet; analyzing the sample in the analytical toilet to detect the one or more antibodies; determining the status of the user with respect to the one or more antibodies based on the result of the analysis; and submitting to an authority the identity of the user and the status of the user with respect to the antibodies detected for the purpose of obtaining clearance from the authority.

Further aspects and embodiments are provided in the foregoing drawings, detailed description, and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 illustrates an analytical toilet with the lid closed, according to an embodiment of the disclosure.

FIG. 2 illustrates an analytical toilet with lid open, according to an embodiment of the disclosure.

FIG. 3 illustrates an analytical toilet with lid closed and a portion of the exterior shell removed, according to an embodiment of the disclosure.

FIG. 4A illustrates a user entering identification information manually at an analytical toilet system, according to an embodiment of the disclosure.

FIG. 4B illustrates a user entering identification information using a QR code at an analytical toilet system, according to an embodiment of the disclosure.

FIG. 4C illustrates a user entering identification information biometrically at an analytical toilet system, according to an embodiment of the disclosure.

FIG. 5 illustrates the various steps of a method to obtain travel clearance, according to an embodiment of the disclosure.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” refers to the portion of the toilet below and around the bowl supporting it.

As used herein, the term “user” refers to any individual who interacts with the toilet and deposits excreta therein.

As used herein, the term “excreta” refers to any substance released from the body including urine, feces, menstrual discharge, saliva, mucus, expectorate, sputum, and anything contained therein or excreted therewith.

As used herein, the term “sputum” refers to a mixture of saliva and mucus coughed up from the respiratory tract, typically as a result of infection or other disease and often examined to aid medical diagnosis.

As used herein, the term “manifold” is intended to have a relatively broad meaning, referring to a device with multiple conduits and valves to controllably distribute fluids, namely water, liquid sample and air.

As used herein, the term “test chamber” is meant to refer broadly to any space adapted to receive a sample for testing, receive any other substances used in a test, and apparatus for conducting a test, including any flow channel for a fluid being tested or used for testing.

As used herein, the term “sensor” is meant to refer to any device for detecting and/or measuring a property of a person or substance regardless of how that property is detected or measured, including the absence of a target molecule or characteristic.

As used herein, the term “microfluidics” is meant to refer to the manipulation of fluids that are contained to small scale, typically sub-millimeter channels. The “micro” used with this term and others in describing this invention is not intended to set a maximum or a minimum size for the channels or volumes.

As used herein, the term “microfluidic chip (MFC)” is meant to refer to is a set of channels, typically less than 1 mm², that are etched, machined, 3D printed, or molded into a microchip. The micro-channels are used to manipulate microfluidic flows into, within, and out of the microfluidic chip.

As used herein, the term “microfluidic chamber” is meant to refer to a test chamber adapted to receive microfluidic flows and/or a test chamber on a microfluidic chip.

As used herein, the term “lab-on-chip” is meant to refer to a device that integrates one or more laboratory functions or tests on a single integrated circuit. Lab-on-a-chip devices are a subset of microelectromechanical systems (MEMS) and are sometimes called “micro total analysis systems” (μTAS).

As used herein, the term “data connection” and similar terms are meant to refer to any wired or wireless means of transmitting analog or digital data and a data connection may refer to a connection within a toilet system or with devices outside the toilet.

As used herein, “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, “analyte” is meant to refer to a substance whose chemical constituents are being identified and measured.

As used herein, the prefix “nano-” is meant to refer to something in size such that units are often converted to the nano-scale for ease before a value is provided. For example, the dimensions of a molecule may be given in nanometers rather than in meters.

As used herein, “miniaturized electronic system” is meant to refer to an electronic system that uses nanometer scale technology.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, a “authority” is meant to refer to a governing body. The governing body can be a government of a nation or governmental agency of a nation or a non-governmental agency. The governing body can preside over a form of public transportation such as an airline, subway, bus, ship, cruise line, city or regional transit, privately contracted and operated transit or other transportation organization. The governing body can preside over a publicly or privately held sports or athletics organization. The governing body may be made up of one or more people. The governing body can establish rules, guidelines or laws that a user must follow in order for a user to use or have access to the services provided.

As used herein, “clearance” is meant to refer to allowance granted to a user who does not have an active infectious disease and further has antibodies to one or more infectious diseases. The clearance allows the user to use public transportation, travel across borders, pass through ports of entry, attend amusement parks, attend or participate in sporting events, return to work, enter a public facility or in general, participate in activities where large groups of people congregate. The user may also include employees who work for public transportation, amusement parks, sports, border control, ports of entry or other organizations that manage and come into contact with large groups of people. A user who has obtained post-infection clearance may also be referred to as one who has been “certified”, “granted permission” or “approved”. In particular, a user may receive an “immunity certificate”.

As used herein, “FET” is meant to refer to a field effect transistor, which is a device which uses an electric field to control the current flowing through a device. FETs are also known by the name “unipolar transistor”.

As used herein, a “NAT” is meant to refer to a nucleic-acid test which is a technique used to detect a particular nucleic acid sequence and thus usually to detect and identify a particular species or subspecies of organism, often a virus or bacteria that acts as a pathogen in blood, tissue, urine, etc. NATs differ from other tests in that they detect genetic materials (RNA or DNA) rather than antigens or antibodies. Detection of genetic materials allows an early diagnosis of a disease because the detection of antigens and/or antibodies requires time for them to start appearing in the stool, bloodstream, or other locations.

As used herein, a “NAAT” is meant to refer to a nucleic-acid amplification test to identify small amounts of DNA or RNA in test samples. They can, therefore, be used to identify bacteria, viruses, and other pathogens even when the material of interest is present in very small amounts. NAATs required an additional step to amplify the genetic material by making copies of it. NAATs are typically used in conjunction with such amplification methods as PCR, strand displacement assay (SDA), or transcription mediated assay (TMA).

As used herein, the term “virus” is given its ordinary meaning, namely a small infectious agent, comprised of genetic material within a capsid (protein coat), that replicates only inside the living cells of an organism.

As used herein, the term “antigen” is meant to refer to a toxin or other foreign substance which induces an immune response in the body, especially the production of antibodies. Antigens are “targeted” by antibodies wherein each antibody is specifically produced by the immune system to match an antigen after cells in the immune system come into contact with the antigen.

As used herein, the term “antibody” (also known as an immunoglobulin (Ig)) is meant to refer to a large, Y-shaped protein produced mainly by plasma cells that is used by the immune system to neutralize pathogens, such as pathogenic bacteria and viruses. Classes of human antibodies include IgA, IgD, IgE, IgM and IgG.

As used herein, the term “immunoglobulin M” (IgM) is one of several classes of antibody isotypes that are produced by vertebrates. IgM is the largest antibody, and it is the first antibody to appear in the response to initial exposure to an antigen, such as a pathogenic virus or bacteria. For example, humans start producing IgM antibodies against the coronavirus around 10 days after showing symptoms (perhaps 15 days after infection).

As used herein, the term “immunoglobulin G” (IgG) is one of several antibody isotypes that are produced by humans. IgG is the main type of antibody found in blood and extracellular fluid, allowing it to control infection of body tissues. By binding many kinds of pathogens such as viruses, and bacteria, IgG protects the human body from infection. For example, after about twelve days after infection by a coronavirus, humans start making IgG antibodies, and gradually stop making IgM. Most individuals will recover fully from the coronavirus as soon as IgG levels ramp up. If IgM antibodies are present in the individual's blood, the individual is likely to still be infected. If only IgG is present, the individual is recovering or fully recovered.

As used herein, a “coronavirus” is a type of virus that causes diseases in birds and mammals. In humans, coronaviruses cause respiratory tract infections that can be mild, such as some cases of the common cold, and others that can be lethal. The coronavirus class of viruses includes alphacoronavirus, betacoronavirus, gammacoronavirus, or deltacoronavirus. More specifically, these viruses include severe acute respiratory syndrome (SARS-CoV), SARS-CoV-2 (also known as COVID-19), and middle east respiratory syndrome (MERS-CoV).

As used herein, the term “recombinant protein” is a manipulated form of protein, which is generated in various ways to produce large quantities of proteins, modify gene sequences and manufacture useful commercial products. The formation of recombinant protein is carried out in specialized vehicles known as vectors. Recombinant technology is the process involved in the formation of recombinant protein.

As used herein, the term “immunoassay” is a biochemical test that measures the presence or concentration of a macromolecule or a small molecule in a solution typically through the use of an antibody. In some instances, an antigen may be used. In addition to the binding of an antibody to its antigen, the other key feature of all immunoassays is a means to produce a measurable signal in response to the binding. This typically includes chemically linking antibodies or antigens with some kind of detectable label. The labels are detectable as they usually either emit radiation, produce a color change in a solution, fluoresce under light, or can be induced to emit light.

As used herein, the term “lateral flow assay” (LFA) also known as a lateral flow immunochromatographic assay is a simple cellulose-based device intended to detect the presence of a target analyte in a liquid sample without the need for specialized and costly equipment. Lateral flow tests operate on the same principles as the enzyme-linked immunosorbent assays (ELISA). LFA tests run a liquid along the surface of a pad with reactive molecules that show a visual positive or negative result. Typically, these tests are used for medical diagnostics for home testing, point of care testing, or laboratory use.

As used herein, the term “enzyme-linked immunosorbent assay” (ELISA) is a commonly used analytical biochemistry assay. The assay uses a solid-phase enzyme immunoassay to detect the presence of a ligand (commonly a protein) in a liquid sample using antibodies directed against the protein to be measured. In the simplest form of an ELISA, antigens from the sample are attached to a surface. A matching antibody is applied over the surface so it can bind to the antigen. This antibody is linked to an enzyme, and in the final step, a substance containing the enzyme's substrate is added. The subsequent reaction produces a detectable signal, most commonly a color change.

As used herein, the term “western blot” is a widely used analytical technique in molecular biology and immunogenetics to detect specific proteins in a sample of tissue homogenate or extract. A western blot may be used to detect antibodies. The western blot method is composed of a gel electrophoresis to separate native proteins by 3-D structure or denatured proteins by the length of the polypeptide, followed by an electrophoretic transfer onto a membrane (such as PVDF or Nitrocellulose) and an immunostaining procedure to visualize a target protein on the blot membrane.

### Exemplary Embodiments

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

One particular variety of detection, analysis, and trend tracking is related to biomarkers. A bio-marker, or biological marker is a measurable indicator of some biological state or condition. Biomarkers are often measured and evaluated to examine normal biological processes, pathogenic processes, or pharmacologic responses to a therapeutic intervention. Biomarker information can be a valuable resource in providing for the health and wellness of an individual or population. Some of the uses include being used to detect a contagious disease at its earliest stages and further monitoring the formation of antibodies during and after an infection of the contagious disease.

There can be significant risk to the general public when in close contact with individuals who are infected with a contagious disease, such as a coronavirus. Individuals who may or may not be showing symptoms may be unaware they are infected with a contagious disease and are acting as a disease vector. Closed and confined areas such as airplane cabins, buses, commuter trains and subway cars are especially robust areas to spread diseases. Nevertheless, individuals who have been infected and recovered from an infectious disease should, ideally, be allowed to be in close contact with the general public, even in an epidemic or pandemic, as long as the individual has antibodies against the disease, i.e., has acquired immunity against the disease.

The present disclosure relates to a method to obtain clearance, in particular post-infection clearance. The disclosure further relates to analytical toilets with analytical tools (may also be referred to as a “smart toilet” or a “health and wellness” toilet) which qualitatively or quantitatively detects, analyzes, and/or tracks the trends of analytes, such as biomarkers, of a user who deposits excreta into the toilet. More specifically, the toilet receives excreta or sputum from a user, processes the excreta or sputum in preparation for analysis, and brings a sample extracted from the excreta or sputum into a testing area for detection of one or more antibodies by a detection system. In some embodiments, sputum deposited upon a probe may also be deposited in the toilet or inserted into a receptacle for analysis. The antibodies may be from an active or non-active infection of a contagious disease from one or more viral or bacterial antigens. In particular, the detection system can detect antibodies caused by infections from bacteria or viruses such as alphacoronavirus, betacoronavirus, gammacoronavirus, deltacoronavirus, SARS-CoV, SARS-CoV-2, MERS-CoV, influenza A, influenza B, influenza C, Ebola, dengue, viral meningitis, Zika, hepatitis A-E, tuberculosis, tetanus, typhoid fever, typhus, leprosy, diphtheria, streptococcus, staphylococcus, pertussis, chlamydia, bacterial meningitis, mycoplasma pneumonia, and any related variants.

The detection area (i.e., detection chamber) may comprise an immunoassay, NAT, NAAT, western blot, or FET-based detection system. Preferably, especially when detecting antibodies, the detection system uses an immunoassay, such as immunoagglutination (also known as immunoagglomeration or immunopreciptation), immunoblotting (e.g., lateral flow assay “LFA”), immunosorbent (e.g., enzyme-linked immunosorbent assay “ELISA”) or an indirect enzyme immunoassay (EIA). Such immunoassays preferably use labels, such as particle labels, fluorescent labels, radiolabels, and dye labels.

After the toilet has finished with the sample, the toilet purges the sample from the toilet in preparation for receiving a new sample of excreta or sputum.

The information received from the detection system in the analytical toilet may be used by an authority to grant clearance to a user if the user is found to have recovered from an infectious disease even in an epidemic or pandemic. The status of the user with respect to the one or more antibodies is indicative that the user has acquired an immunity to the infection. For example, clearance would not be granted to a user who has not been infected by a contagious disease or may have an active infection of a contagious disease that is causing an epidemic or pandemic. This would prevent the user from crossing continental, national, state, city, county, region, or other borders and would prevent the user from using one or more forms of public transportation, pass through ports of entry or generally be in areas where large groups of people congregate.

In various exemplary embodiments, the analytical toilet comprises test devices that are designed to perform one or more of a variety of laboratory tests. Any test that could be performed in a medical or laboratory setting may be implemented in an analytical test device. These tests may include measuring pulse, blood pressure, blood oxygenation, electrocardiography, body temperature, body weight, excreta or sputum content, excreta or sputum weight, excreta or sputum volume, excreta or sputum temperature, excreta or sputum density, excreta flow rate, and other health and wellness indicators in addition to antibody detection. This information may also be used to grant or not grant clearance to a user. In particular, all information that is gathered in the natural course of a user availing themselves of the toilet system may also be used to grant or not grant clearance. The information may be gathered in a passive or active manner.

In accordance with the present disclosure, an analytical toilet that includes an infrastructure for multiple health and wellness analysis tools is provided. This provides a platform for the development of new analytical tools by interested scientists and companies. Newly developed tests and diagnostic tools may be readily adapted for use in a system having a consistent tool interface.

In various exemplary embodiments, the analytical toilet provides a fluid processing manifold that collects and routes samples from the toilet bowl to various scientific test devices and waste handling portals throughout the device.

In various exemplary embodiments, the analytical toilet provides multiple fluid sources via a manifold system. The manifold is adapted to connect to a plurality of analytic test devices adapted to receive fluids from the manifold. The manifold is designed to selectively provide a variety of different fluid flows to the analytical test device. These fluids may include, among others, excreta samples, sputum, buffer solutions, reagents, water, cleaners, biomarkers, dilution solutions, calibration solutions, and gases such as air or nitrogen. These fluids may be provided at different pressures and temperatures. The manifold and analytical test device are also adapted to include a fluid drain from the analytical test devices.

In various exemplary embodiments, the manifold system provides a standardized interface for analytical test devices to connect and receive all common supplies (e.g., excreta samples, sputum, flush water), data, and power. Common supplies may be supplied from within (e.g., reagents, cleaners) or without (e.g., water) the toilet system. The analytical test devices may be designed to receive some or all of the standardized flows. The analytical test devices may also include storage cells for their own unique supplies (e.g., test reagent).

In various exemplary embodiments, the manifold is adapted to direct fluids from one or more sources to one or more analytical test devices. The manifold and analytical test devices are designed such that analytical test devices can be attached to and detached from the manifold making them interchangeable based on the needs of the user. Different analytical test devices are designed to utilize different test methods and to test excreta or sputum samples for different antibodies as a result of an infection from a contagious bacteria or virus.

In various exemplary embodiments, the analytical toilet provides an electrical power connection and a data connection for the analytical test device. In a preferred embodiment, the electrical power and data connections use the same circuit. In various exemplary embodiments, the toilet is provided with pneumatic and/or hydraulic power to accommodate the analytical test devices.

In various exemplary embodiments, the analytical toilet platform performs various functions necessary to prepare samples for examination. These functions include, but are not limited to, separating urine from feces, diluting, or concentrating samples, large particle filtration, sample agitation, and adding reagents.

In various exemplary embodiments, the analytical toilet also provides, among other things, fluid transport, fluid metering, fluid valving, fluid mixing, separation, amplification, storage and release, and incubation. The analytical toilet also is equipped to provide cleansers, sanitizers, rinsing, and flushing of all parts of the system to prevent cross-contamination of samples. In some embodiments, the system produces electrolyzed water for cleaning.

In various exemplary embodiments, one layer of the fluidic manifold is dedicated to macro-scale mixing of fluids. Sample, diluents, and reagents are available as inputs to the mixers. The mixing chamber is placed in series with all other scientific test devices, allowing bulk mixed sample to be routed to anywhere from one to all stations (i.e., analytical test device interfaces) for analysis. Mixing may also occur in an analytical test device.

In various exemplary embodiments, urine samples are filtered for large particulates at the fluid ingress ports of the manifold. The fluid manifold uses a network of channels along with simple valves to route prepared stool samples to one of several scientific test devices located on the platform.

In various exemplary embodiments, the manifold is constructed using additive layers, and different layers can be customized for particular applications. Standard ports and layouts are used for interfacing with external components, such as pressure sources and flow sensors. In general, characteristic channel volumes at the bottom of the manifold stack are on the order of milliliters. At the top of the manifold stack is the microfluidic science device, which will interface simultaneously with multiple microfluidic chips using standardized layout and pressure seals.

In various exemplary embodiments, the system is adapted to work with a variety of actuation technologies that may be used in the analytical test devices. The system provides electronic and fluidic interconnects for various actuator technologies and supports OEM equipment. In a preferred embodiment, the system is adapted to work with actuator modules that can be attached to the sample delivery manifold and controlled by a central processor. The system platform supports an inlet and outlet for the pressure transducer that interfaces with the fluidic manifold, and electronic or pneumatic connections where required. The system supports a variety of macro- and microfluidic actuation technologies including, but not limited to, pneumatic driven, mechanical pumps (e.g., peristaltic), on-chip check-valve actuators (e.g., piezo-driven or magnetic), electroosmotic driven flow, vacuum pumps, and capillary or gravity driven flow (i.e., with open channels and vents).

One benefit of the present disclosure is the detection of antibodies without having any inconvenience aside from what they would otherwise have using the toilet. Without the present disclosure, among other things, people often must manually collect samples of excreta, use equipment they are less familiar with than a toilet, or wait longer for analysis and results. Each of these things can negatively impact a user's experience and/or the quality or accuracy of the results. Additionally, the present disclosure describes tracking the antibody trend data of a population of users. This may be particularly beneficial in the detection of an emergence and current status of an epidemic or even a pandemic.

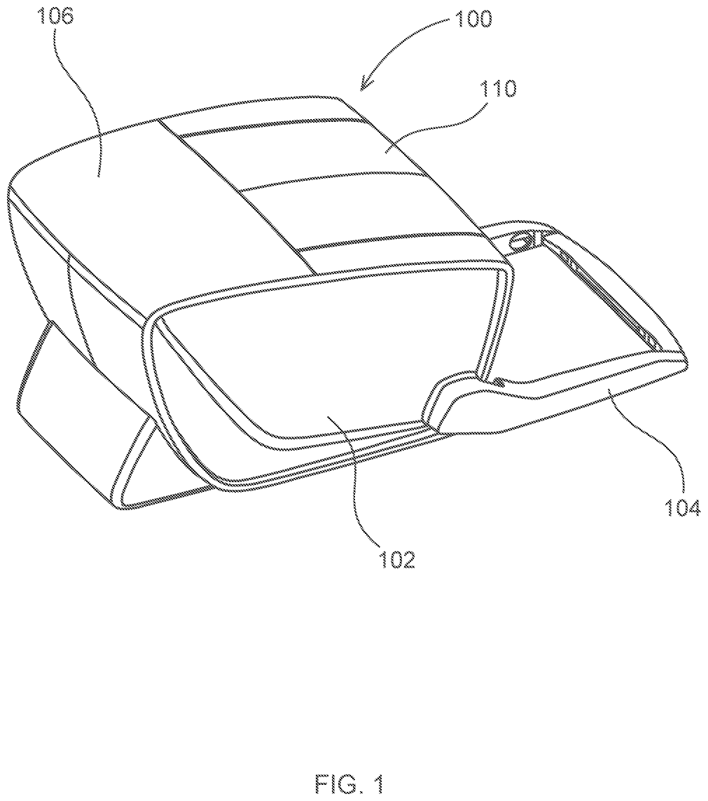

Now referring to FIGS. 1-3, a preferred embodiment of an analytical toilet 100 is shown. FIG. 1 illustrates the analytical toilet 100 with the lid 110 closed, according to an embodiment of the disclosure. FIG. 1 further shows exterior shell 102, foot platform 104 and rear cover 106. The lid 110 is closed to prevent a user from depositing urine in toilet 100 until the toilet is ready for use.

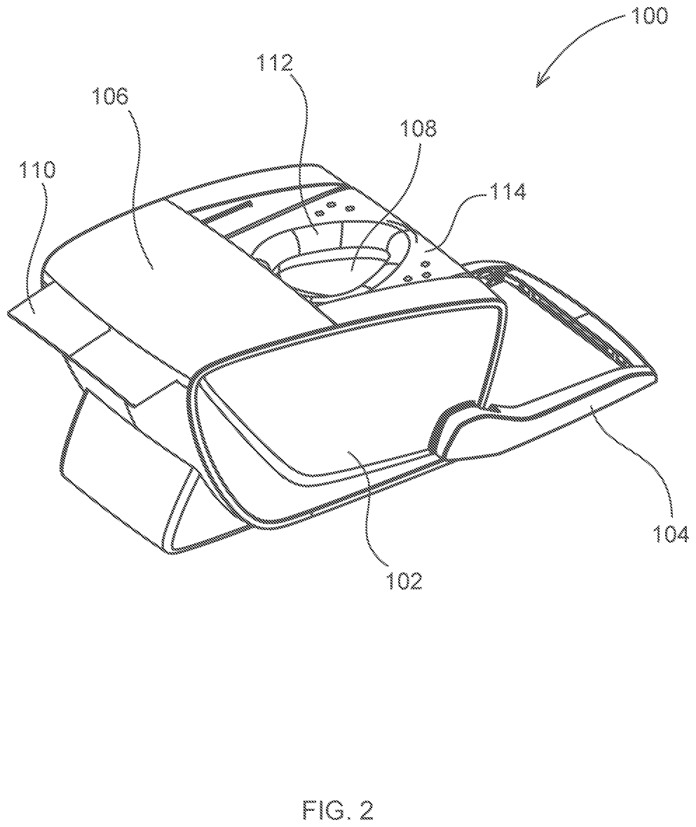

FIG. 2 illustrates toilet 100 with lid 110 open, according to an embodiment of the disclosure. Toilet 100 includes exterior shell 102, rear cover 106, bowl 108, seat 112, lid 110, fluid containers 114 and foot platform 104. Housed within toilet 100 are a variety of features, including equipment, that facilitate receiving excreta, processing urine for analysis, analyzing urine, and disposing of urine. FIG. 2 shows toilet 100 with lid 110 open so a user can sit on seat 112 and deposit urine in toilet 100.

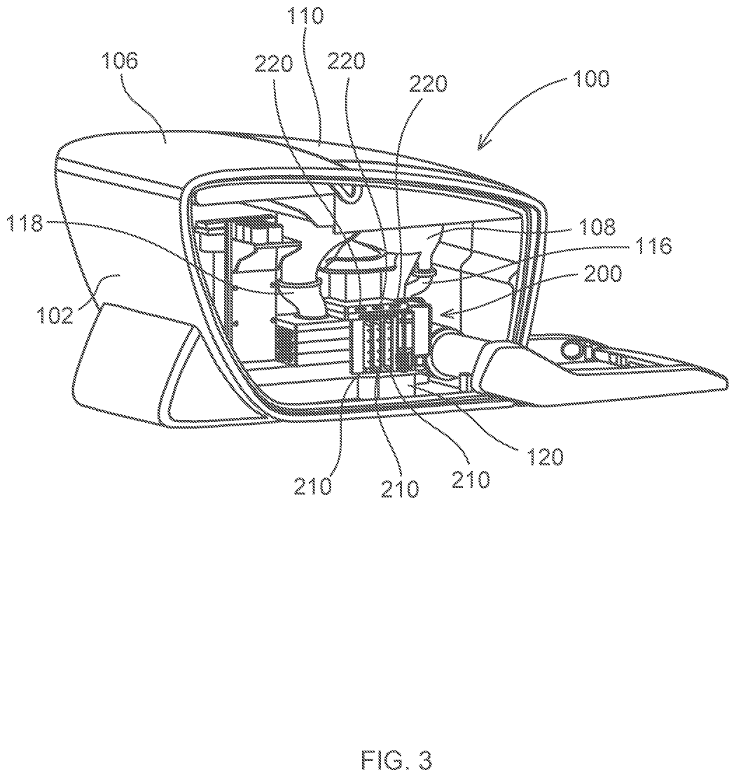

FIG. 3 illustrates toilet 100 with lid 110 closed and a portion of exterior shell 102 removed, according to an embodiment of the disclosure. This allows access to equipment housed within toilet 100. With exterior shell 102 removed, base 120, urine collection pipe 116, feces collection pipe 118, and manifold area 200 is visible. Urine collection pipe 116 further comprises a passageway to deliver a urine sample to the manifold area 200 and to a detection system. Manifold area 200 includes test areas 210 and fluidic chip slots 220. Preparation and/or analysis of sample can selectively take place in a test area 210 or fluidic chip slot 220. Manifold area 200 is the area where analysis takes place. A filter may be added over the entrance of the urine collection pipe 116 to prevent solid material, such as feces or toilet paper, from entering the pipe.

A manifold 200 is located below the bowl 108. The manifold 200 comprises a plurality of fluid paths. These fluid paths allow the manifold 200 to move fluids between the bowl 108, fluid containers 114, outside sources (e.g., municipal water supplies), other sources (e.g., air or water electrolyzing unit), analytical test devices 210, and the toilet outlet. The analytical test devices 210 make up a detection system for one or more viruses. The manifold 200 also provides electrical power and data connections to the analytical test devices 210. The manifold 200 can also directly pass fluids and/or solids from the bowl 108 to the toilet outlet. The manifold 200 is adapted to provide receptacles 210 with standardized connection interfaces for multiple analytical test devices 210. The manifold 200 is shown here with multiple fluid sources 220 for the analytical test device 210. In various embodiments, the manifold 200 may include receptacles for more than one type of analytical test device 210 (e.g., different sizes, fluid supply needs, etc.). In various exemplary embodiment, the analytical test device 210 includes multiple inlets in fluid communication with the manifold 200. The analytic test device 210 may also include at least one outlet or drain in fluid communication with the manifold 200.

In various exemplary embodiments, manifold 200 may comprise a microfluidic system to isolate and transport a sample, add and mix reagents if appropriate, filter out solids, and test the sample for one or more coronaviruses on a small scale (i.e., sub-millimeter scale) in a health and wellness analytical toilet described herein. The microfluidic system may comprise an open microfluidic system, continuous-flow microfluidic system, droplet-based microfluidic system, digital microfluidic system, nanofluidic system, paper-based microfluidic system or combinations thereof. The microfluidic system may be used to introduce and feed fluidic samples into paper-based immunoassay test strips, cartridges or cassettes. The paper-based test strips, cartridges or cassettes may be for single-use (i.e., one time use) only.

In one embodiment of a method using one-time use assay cartridges, the analyzer in an analytical toilet comprises a plurality of cartridges, each configured to run a one-time immunoassay, and an aligning mechanism configured to align a single cartridge with the sample to be analyzed for each excreta event.

The system of claim 41, wherein the aligning mechanism is configured to move the single cartridge into alignment with a port through which the sample enters the cartridge and into alignment with a sensor to detect the result of the one-time immunoassay.

A microfluidic-based antibody detection system may be located on a microfluidic chip (MFC). In a preferred embodiment, the MFC includes a test chamber with a lab-on-chip (“LoC”) (also known as “test-on-chip”). The LoC may be designed to perform one or more laboratory tests. In various exemplary embodiments, one or more microfluidic chips (MFCs) may be removed or added to the toilet system as desired or needed at any given time, such as for different antibody tests. In exemplary embodiments, an immunoassay, such as a lateral flow chromatographic immunoassay (also referred to as a Lateral Flow Assay (LFA)) microfluidic chip may be used as a component in an antibody sensor in a health and wellness analytical toilet. This test is similar to an enzyme-linked immunosorbent assay (ELISA) which may also be used in an MFC.

The LFA chip may comprise an array of immobilized antigens conjugated with colloidal gold or other detectable taggants, such as the qualitative Cellex qSARS-CoV-2 IgG/IgM Rapid Test (Cellex, Research Triangle Park, N.C., USA), that conjugate to antibodies. The LFA further comprises anti-human IgM, anti-human IgG and control goat anti-rabbit IgG. In some embodiments, an analytical toilet described herein may be designed such that a Cellex qSARS-CoV-2 IgG/IgM Rapid Test cassette (also referred to as a “cartridge”) may be inserted into a slot or location in the microfluidic array wherein the analytical toilet feeds a processed sample into the cassette. The test would be adapted to test for antibodies in urine and sputum. Preferably, the cassette is changed out after each test automatically by a mechanism within the analytical toilet. The used cassette may then be ejected into a biohazard container that can be changed out periodically.

Alternatively, the antibody detection system makes use of an immunoagglutination type assay. Immunoagglutination (also known as immunoagglomeration or immunopreciptation) assay may include a latex immunoagglutination assay, piezoelectric immunoagglutination assay or a nephelometric immunoagglutination assay. Techniques used for the detection and quantification of antibodies in an immunoagglutination-based assay include visual observation, light scattering, turbidimetry, nephelometry, and angular anisotropy.

In one embodiment, the antibody detection system comprises an analysis chamber, a sample mechanism to add the sample to the analysis chamber, a reagent mechanism to add an immunoagglutination reagent to the chamber, a sensor to detect agglutination in the analysis chamber, indicating the presence of the one or more antibodies in the sample, and a flush mechanism to wash the analysis chamber after each analysis.

The antibody detection system can make use of immunoblotting (e.g., lateral flow assay “LFA”, western blot) or immunosorbent (e.g., enzyme-linked immunosorbent assay “ELISA”, indirect enzyme immunoassay (EIA) or sandwich ELISA). Other related techniques include dot blot analysis, quantitative dot blot, immunohistochemistry, immunofiltration assays, immunochromatographic assays and immunocytochemistry. Such immunoassays preferably use labels, such as particle labels, fluorescent labels, radiolabels and dye labels.

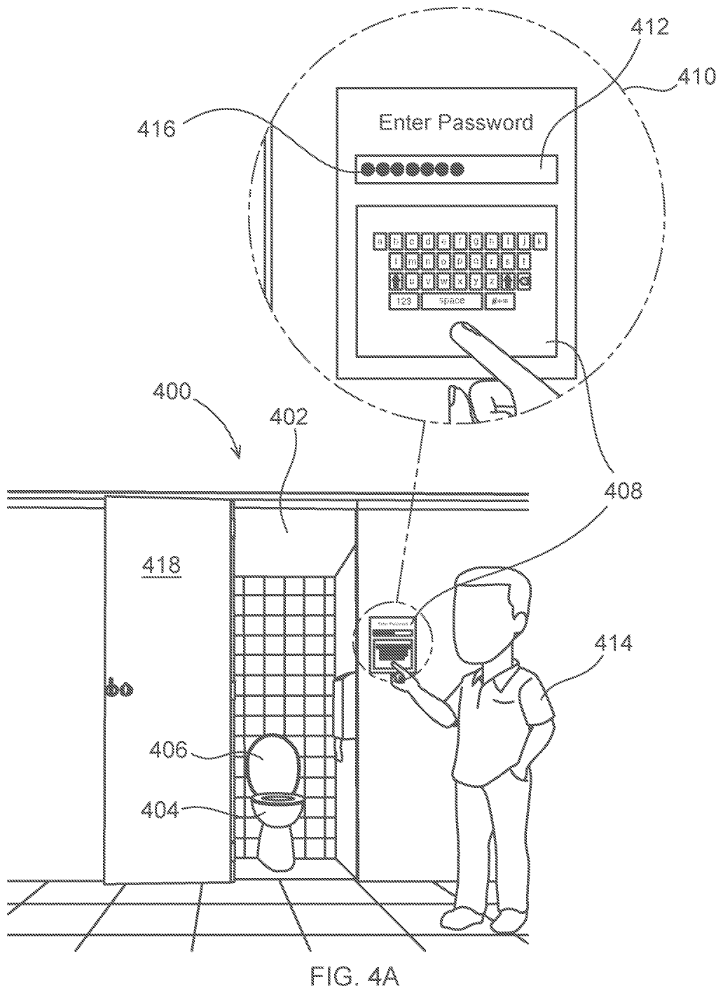

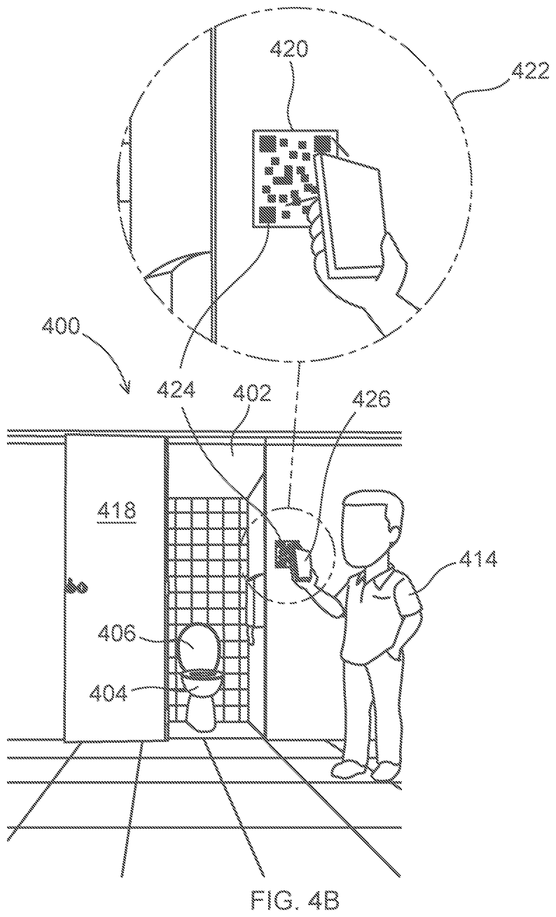

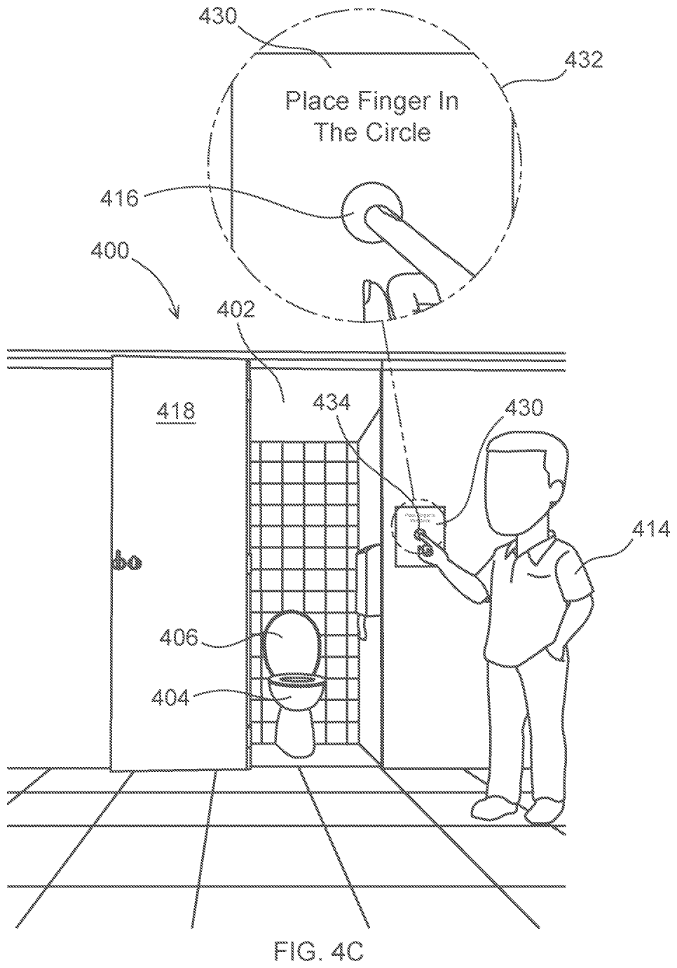

The antibody detection system may comprise an indirect enzyme immunoassay (EIA) which is similar to an ELISA. In an indirect EIA, antigen-specific antibody is quantified rather than the antigen. Indirect EIAs can detect antibodies against many types of pathogens. There are three important differences between indirect and direct ELISAs as shown in FIG. 4.

Indirect EIAs start with attaching known antigen (e.g., spike proteins from a coronavirus) to the bottom of microtiter plate wells. After blocking the unbound sites on the plate, a user's sample is added. If antibodies are present (IgM or IgG), they will bind to the coronavirus spike protein antigen. After washing away any unbound proteins, a secondary antibody with its conjugated enzyme is directed against the primary antibody (IgM or IgG). The secondary antibody allows the analytical toilet to quantify how much antigen-specific antibody is present in the user's urine or sputum by the intensity of the color produced from the conjugated enzyme-chromogen reaction.

An analytical toilet descried herein may comprise an MFC capable of flow cytometry. In this process, a sample containing antibodies is suspended in a fluid and injected into the flow cytometer instrument. The sample is focused to ideally flow one cell at a time through a laser beam, where the light scattered is characteristic to the antibodies and their components. Antigens may be labeled with fluorescent markers, so light is absorbed and then emitted in a band of wavelengths as the antigen binds with antibody. The flow cytometer analyzer can provide quantifiable antibody data from a sample.

An analytical toilet descried herein may comprise an MFC capable of integrating ELISA, western blot or LFA testing capability that further comprises a recombinant protein. The recombinant protein may be used as an antigen to bind to and detect antibodies. The recombinant protein may be any part or portion of a contagious bacteria or virus in which a user has antibodies against and are found in excreta. For example, a recombinant protein of a complete or portion of a SARS-CoV-2 spike protein or a SARS-CoV-2 nucleocapsid protein may be used as an immobilized antigen in an MFC ELISA or LFA test to bind to and detect SARS-CoV-2 antibodies in a sample of excreta. The nucleocapsid recombinant proteins and spike recombinant proteins (RayBiotech Life, Peachtree Corners, Ga., USA) listed in Table 1 may be used in an ELISA, western blot or LFA antibody test device configured in an analytical toilet described herein. Although only SARS-CoV-2 recombinant proteins are listed in Table 1, recombinant proteins from portions of other infectious bacteria and virus-based diseases may also be used.

TABLE 1
Nucleocapsid and Spike Recombinant Proteins

| | | | Expression | | | Molecular |
|---|---|---|---|---|---|---|
| Protein | Catalog# | Protein Domain | Host | Expression Region | Tag | Weight (kDa) |
| N Protein, | 230-30164 | Full Length | HEK293 | Met1 - Ala419 | C-terminal His-tag | ~50 |
| Nucleocapsid | 230-01104 | | E. Coli | Met1 - Ala419 | N-terminal His-tag | ~50 |
| | 230-20409 | | HEK293 | Met1 - Ala419 | C-terminal His-tag | ~50 |
| S Protein, | 230-01102 | S1 Subunit, RBD | E. Coli | Arg319 - Phe541 | N-terminal His-tag | ~25 |
| Spike | 230-30162 | | HEK293 | Arg319 - Phe541 | C-terminal His-tag | ~25 |
| | 230-20406 | | HEK293 | Arg319 - Phe541 | C-terminal His-tag | ~25 |
| | 230-20405 | | HEK293 | Arg319 - Phe541 | C-terminal Fc-tag | ~50 |
| | 230-01101 | S1 Subunit, Full | E. Coli | Val16 - Gln690 | N-terminal His-tag | ~75 |
| | 230-20407 | Length | HEK293 | Val16 - Gln690 | C-terminal His-tag | ~75 |
| | 230-01103 | S2 Subunit, Full | E. Coli | Met697 - Pro1213 | N-terminal His-tag | ~58 |
| | 230-30163 | Length | HEK293 | Met697 - Pro1213 | C-terminal His-tag | ~80 |
| | 230-20408 | | HEK293 | Met697 - Pro1213 | C-terminal His-tag | ~60 |

In various exemplary embodiments, an MFC is designed to use very small quantities of reagent. In various exemplary embodiments, reagents are dispensed using technology similar to that used in inkjet printers to dispense ink. In some embodiments, an electrical current is applied piezoelectric crystal causing its shape or size to change forcing a droplet of reagent to be ejected through a nozzle. In some embodiments, an electrical current is applied to a heating element (i.e., resistor) causing reagent to be heated into a tiny gas bubble increasing pressure in the reagent vessel forcing a droplet of reagent to be ejected.

In various exemplary embodiments, the toilet fluidic manifold 200 provides routing. Interconnecting levels of channels allows routing from one port to all others. Each channel includes an accumulator; allows for constant pressure pumping of all active channels simultaneously, while time-multiplexing pump-driven inflow.

In various exemplary embodiments, the manifold has reaction chambers built in for general purpose mixing and filtering operations. Each chamber has a macro-sized channel through which the manifold delivers a sample extracted from urine (filling the reaction chamber), and the chamber has a micro-sized channel. Pumps located internal or external to the manifold drive fluid into the reaction chamber, and into the micro-sized channel. A valve at the output of the macro-channel, and possibly at the output of the micro-channel, controls fluid direction as it exits the reaction chamber.

Microfluidic applications require support infrastructure for sample preparation, sample delivery, consumable storage, consumable delivery or replenishment, and waste extraction. In various exemplary embodiments, the manifold includes integrated support for differential pressure applications, pneumatic operations, sample and additive reservoirs, sample accumulators, external pumps, pneumatic pressure sources, active pump pressure (e.g., peristaltic, check-valve actuators, electro-osmotic, electrophoretic), acoustic or vibrational energy, and light-interaction (e.g., spectrometer, laser, UV, magnification). The acoustic energy source may be a high frequency (54 MHz) bulk acoustic wave (BAW) actuator.

In various exemplary embodiments, the manifold interface has a matrix of ports, possibly laid out in a regular grid. These ports may be activated or closed via an external support manifold. Routing is fully programmable.

In various exemplary embodiments, the manifold 200 directs one or more fluids to the analytical test device 210 or an MFC analytical test device to cleanse the devices. These may include cleaning solutions, disinfectants, detergents, and flushing fluids. In various exemplary embodiments, the manifold directs hot water or steam to clean sample, reagents, etc. from the devices. In various exemplary embodiments, the toilet systems using oxygenated water, ozonated water, electrolyzed water, which may be generated on an as-needed basis by the toilet system (this may be internal or external to the toilet).

In some embodiments, the manifold 200 comprises a heater. The heater may be used to drive off water and concentrate a urine sample.

In various exemplary embodiments, waste from the MFCs is managed based on its characteristics and associated legal requirements. Waste that can be safely disposed is discharged into the sewer line. Waste that can be rendered chemically inert (e.g., heat treatment, vaporization, neutralization) is processed and discharged. Waste that cannot be discharged or treated in the toilet system is stored, and sequestered if necessary, for removal and appropriate handling.

In various exemplary embodiments, the manifold creates sequestered zones for each of these waste categories and ensures that all products are properly handled. In various exemplary embodiments, the manifold directs flushing water and/or cleansing fluids to clean the manifold and MFC. In some embodiments, high-pressure fluids are used for cleaning. In such an embodiment, the high-pressure fluids are not used in the MFC. In some embodiments, the MFC is removed from the backplate interface and all ports are part of the high-pressure cleansing and/or rinse.

There are many ways to incorporate antibody detection systems into the toilet, the selection of which will depend on various factors, including ease of manufacture and maintenance, target market, physical constraints, frequency of use compared to other desired functions of the toilet, and cost. In one preferred embodiment, the detection system is integrated with a fluidic circuit. More preferably, the fluidic circuit is on a fluidic card. Still more preferably, the fluidic circuit on the fluidic card is a microfluidic circuit on a micro fluidic card. Yet more preferably, the microfluidic circuit interfaces with nano-scale fluidic circuits. The fluidic card may be inserted into a slot or receptacle of the toilet which connects the fluid circuit on the card to the toilet's fluidic delivery system, enabling the card to receive the sample derived from excreta or sputum. Alternatively, the detection system is part of a larger device that may be attached to the toilet, such as a device that processes and/or analyzes the sample extracted from excreta or sputum. Alternatively, the detection system is built into the toilet rather than being on a card. Alternatively, the sensor is external to the remainder of the toilet and is connected to receive and/or return fluid from the toilet, such as may be accomplished by connecting the sensor to a part of the toilet with tubes or pipes.

In some embodiments, analytical toilet 100 may further comprise one or more sensors to collect information such as the temperature, blood pressure, pulse, body weight and electrocardiography (EKG), blood oxygenation, water retention, skin moisture, excreta content, excreta weight, excreta volume, excreta temperature, excreta density, and excreta flow rate of the user. This information may be used in conjunction with antibody tests to determine if a user has an active contagious disease. The sensors may be located in the seat 112, lid 110 or other location in the toilet. To gain an accurate body temperature of a user, an infra-red (IR) camera may be located in the bowl such that the temperature of a stream of urine may be taken as it is leaving the body of the user. This would provide an accurate method of determining the core temperature of the user. In a preferred embodiment, the seat is attached to the toilet via a powered quick disconnect system that allows the seat to be interchangeable. This facilitates installing custom seats to include user-specific tests based on known health conditions. It also facilitates installing upgraded seats as sensor technology improves.

### Method to Obtain Post-Infection Clearance

In a first step in a method to obtain post-infection clearance, a user downloads an application onto their mobile phone, computer or tablet. The user sets up an account using the application over an encrypted network and enters driver's license, passport, social security information and any other information required to prove the user's identity. In another method, a user may purchase a card with a chip or magnetic stripe that contains identification information that may only be used with an analytical toilet.

The user's account may be linked to a governmental authority such as the United States (U.S) Department of State, U.S. Department of the Interior, U.S. Department of Transportation (USDOT), U.S. Homeland Security, U.S. Transportation Security Administration (TSA), U.S. Immigration and Customs Enforcement (ICE), U.S. Border Patrol, Centers for Disease Control and Prevention, or other authority. The user's account may be linked to a non-governmental authority such as a non-governmental organization (NGO). The user's account may also be linked to any foreign authority such as a foreign governmental body, foreign non-governmental body, the United Nations (UN) or other trans-national organization. The user's account may be directly linked to one or more transportation-based authorities. Such accounts may be a ride sharing account such as Uber or Lyft. The accounts may be an airline account such as American Airlines, Allegiant, Southwest, United Airlines, JetBlue, or another domestic or foreign airline. Other accounts may be a passenger train account such as Amtrak, a public or privately operated transit authority account for a local bus system, subway system or ferry system. Other accounts may include a cruise line or passenger ship line such as Carnival Cruise Line or Canadian Star Line. Other accounts may include a cargo shipping line. The user's account may be linked to their health care provider or employer. The user's account may be linked to a location where large groups of people congregate such as amusement parks, hotels, national parks, beaches, convention centers or stadiums. The user's account may be linked to one or more athletic competition authorities to allow or not allow a user to compete in an athletic competition such as wrestling, football, basketball, martial arts, or other sport.

Once an account has been created to identify a user, the user may then locate a certified or approved analytical toilet. The toilet may be linked to any authority described herein or other related authority. The toilet may be located in a transportation terminal such as an airport terminal, bus station, train station, ferry station, cruise line terminal or taxi station. The toilet may be located at home, a nursing home, doctor's office, hospital, or a gymnasium of fitness center. The toilet may be placed at the entrance to a place of employment and all would be entrants desiring to enter the place of employment are required to be tested for the one or more antibodies before being permitted to enter the place of employment. The analytical toilet may be placed at the entrance to a public facility and all would be entrants to the facility are required to be tested for the one or more antibodies before permitted to enter the public facility. The analytical toilet may be placed near an international border and all entrants desiring to cross the international border are required to be tested for the one or more antibodies before being permitted to cross the international border. In general, the toilet may be located in a strategic location where large groups of people can come into contact.

In some embodiments, the toilet may be located in a privately owned and exclusive facility such as a lounge or a spa. The lounge or spa may provide a more comfortable, private and sterile setting to utilize the toilet. The lounge or spa may be sex or gender specific. The lounge or spa may grant memberships and require a fee to use the lounge service. For example, a monthly or yearly fee may be charged.

A user may create an electronic data connection with an analytical toilet and enter the user's identification information. This is such that the data from the testing of excreta or sputum is linked to the user. This may be done by logging in to the analytical toilet system using an interface that is in electronic communication with the analytical toilet. This may be done manually by using a touch pad. FIG. 4A illustrates a user entering identification information manually at an analytical toilet system 400, according to an embodiment of the disclosure. Analytical toilet system 400 may also be referred to as a station. An analytical toilet system 400 generally comprises a secure and private room further comprising an analytical toilet and a unit to identify a user and link the antibody analysis information to the user. In some instances, analytical toilet system 400 may be installed in a porta-potty style unit. Porta-potty units may be installed on a mobile platform such as in an ambulance, truck bed, bus, or recreational vehicle (RV). Ports-potty style units may be used to secure a venue on short notice. The system may further be able to send the information to an authority for clearance. An analytical toilet system 400 may comprise two or more toilets. Analytical toilet system 400 comprises a room 402 with an analytical toilet 404 and lid 406. Analytical toilet system 400 comprises a touchpad unit 408. An exploded view 410 of touchpad 408 further illustrates the touchpad unit 408 and display 412. FIG. 4A further illustrates how a user 414 enters information 416 such as a password on the touchpad to identify the user 414. In FIG. 4A, the touchpad is on the outside of the room 402 comprising the toilet 404. In some instances, when a user approaches a room 402, the door 418 may be closed and locked. This assures that after the toilet 404 is cleaned after use by the previous user, the toilet stays clean and sterile until the next user enters the required information, the door 418 is then automatically unlocked and opens for the next user. In some embodiments, touchpad unit 408 may be inside room 402. An attendant may then unlock the room and allow a single user 414 to enter the room and use the analytical toilet 406. The user 414 may then use the analytical toilet 404 wherein the data from the antibody analysis of the excreta or sputum left by the user 414 is linked to the user. The information may then be sent to an authority for clearance or to a health care provider or be kept confidential.

The linking of a user to an analytical toilet may be done using a Bluetooth connection or using a QR code or other optical or wireless connection to a smart device such as a mobile phone, tablet, or computer of a user. FIG. 4B illustrates a user entering identification information using a QR code at an analytical toilet system 400, according to an embodiment of the disclosure. In this example, a user 414 approaches the interface unit 420. A close-up view of the interface unit 420 is shown in exploded view 422. A QR code 424 appears on the interface unit 420. A user 414 places their smart device 426 close enough to the QR code such that the system 400 receives information and recognizes the use 414. The user 414 may then use the analytical toilet 404 wherein the data from the antibody analysis of the excreta or sputum left by the user 414 is linked to the user. The information may then be sent to an authority for clearance, to a health care provider or be kept confidential by the user.

In some embodiments, the linking of a user to an analytical toilet may be done using a QR code on a transportation ticket of a user. For example, the transportation ticket may be a bus ticket, airplane ticket, train ticket or boarding pass. Once the testing is completed, a ticket or receipt may be created wherein a QR code or other optically readable code may be printed on the ticket or receipt.

The linking may also be done biometrically by using a biometric identification unit, such as a fingerprint scanner, palm scanner, face recognition scanner, hand geometry scanner, iris scanner or retinal scanner to identify a user. FIG. 4C illustrates a user entering identification information biometrically at an analytical toilet system 400, according to an embodiment of the disclosure. In this example, a user 414 approaches the biometric interface unit 430. A close-up view of the interface unit 430 is shown in exploded view 432. Biometric interface unit 430 further comprises an aperture 434 where a user 414 may place their finger such that the biometric interface unit 430 can read the fingerprint of the user 414. The fingerprint may then be used to identify the user 414 in order to link the information from the antibody analysis of excreta or sputum in an analytical toilet 404. The information may then be sent to an authority for clearance or to a health care provider or be kept confidential.

In another method to identify a user and link the results from use of an analytical toilet is to use a card with a magnetic stripe with a card reading unit. Such cards could be a passport, driver's license or a credit card. The magnetic stripe may be on a bus ticket, train ticket, airplane ticket, or boarding pass. In some instances, a user could have a card that is specifically issued by an authority, like a membership card, to use an analytical toilet. The card could have a pre-paid option, such as using the toilet at a lounge or spa instead of in a public location. An advantage of this user identification method is that the user could carry a physical membership card with them and would not need a smart phone to access a station—an option that might be more appealing to an older population. A card reading unit may also be used to pay a fee to use the toilet with a credit card, gift card or debit card.

In some instances, the identity of a user may use one or more methods of the identification methods described herein. For example, a user may need to complete a biometric scan in addition to scanning an identification card comprising a magnetic strip in order to link the results to an analytical toilet.

In some embodiments, a user may desire to not set up an account or link the results. The user may want to use an analytical toilet for their own personal antibody analysis evaluation and information and not send the results to an authority. The results of the analysis may not be stored electronically but instead printed on paper ticket.

Whatever the form of or combination of user identification used, the purpose is not necessarily to lock out users who are not-prequalified. The user identification unit may be used to enable the smart features of the toilet, and identification is necessary to connect the data to a user profile. However, in a preferred embodiment, the analytical toilets may be used and accessed like normal washroom facilities by anyone who chooses not to utilize the other features.

A user may next leave a sample of excreta or sputum in an analytical toilet. The analytical toilet processes and prepares the excreta or sputum sample. The sample may then be transferred using a passage to a detection system. One or more detection systems may be installed in the analytical toilet such as a western blot, LFA or ELISA to qualitatively detect or quantitatively detect antibodies. Other detection systems may be installed such as NAT, NAAT or FET-based detection system to detect infectious bacteria and viruses. The detection system analyzes the sample and other health and wellness data taken by the toilet and determines if the user has not been infected by an infectious disease, has an active infection or has had an infection and has antibodies to the infectious disease. The results would allow or deny a user from obtaining clearance. This includes being infected with a contagious disease such as alphacoronavirus, betacoronavirus, gammacoronavirus, deltacoronavirus, SARS-CoV, SARS-CoV-2, MERS-CoV, influenza A, influenza B, influenza C, Ebola, dengue, viral meningitis, Zika, hepatitis A-E, tuberculosis, tetanus, typhoid fever, typhus, leprosy, diphtheria, streptococcus, staphylococcus, pertussis, chlamydia, bacterial meningitis, mycoplasma pneumonia, and any variants thereof.

If the antibody tests in an analytical toilet does not qualitatively detect or quantitatively detect a pre-determined threshold level (i.e., concentration) of IgM or IgG antibodies to a specific disease, then the user may not obtain clearance as the user does not have immunity to the disease. The pre-determined threshold of antibodies required for clearance may be determined by a medical consensus or a health-related organization. The health-related organization may include the Red Cross, World Health Organization (WHO), a country, city, state or county health organization, a health care professional or local clinic or hospital. If the antibody tests in an analytical toilet detects only IgM antibodies, then the user may not obtain clearance as likely the user has an active infection of the disease that the body has produced IgM antibodies for. If the antibody tests in an analytical toilet detects IgM and IgG antibodies, then the user may also not obtain clearance as likely the user has an active infection of the disease that the body has produced IgM and IgG antibodies for. It should be noted that if both IgM and IgG antibodies are detected, but the ratio of IgG to IgM antibodies (i.e., IgG/IgM) falls within an acceptable range, or if the amount of IgG antibodies are above a pre-determined threshold level, then a user may obtain clearance. For example, if IgG/IgM>2, then this may be an indication that IgM antibodies have decreased enough, and IgG antibodies have increased enough to prove that a user no longer has an active disease and additionally is no longer contagious. The acceptable IgG/IgM ratio would be dependent on the disease. If the antibody tests in an analytical toilet detects only IgG antibodies to a disease, then the user is considered to be substantially recovered and may obtain post-infection clearance. Consequently, the analytical toilet of the invention is preferably adapted to measure both IgM and IgG and calculate the ratio therebetween based on the quantitative data of the antibodies.

In addition to antibody testing, the analytical toilet may also include tests for temperature, blood pressure, pulse, body weight and electrocardiography (EKG), blood oxygenation, water retention, skin moisture, excreta content, excreta weight, excreta volume, excreta temperature, excreta density, and excreta flow rate of the user. In some instances, the user may not have an infection but may have congestive heart failure, or high or low blood pressure or other health condition. In this instance, the user may not obtain clearance to participate in athletic competitions, take an intercontinental flight or enter an amusement park or participate in other risky activities that put themselves in danger of a high likelihood of mortality.

The analytical toilet processes the computer readable data from the antibody detection system and sends the information to the user. In this instance, the information could not be sent to an authority as identification of the user was not set up before the test. This prevents fraudulent activity from testing and analysis of one user being sent in place of another user. This method allows for a user to be tested for their own personal and confidential information. The user may choose to keep the information private and be re-tested using an analytical toilet at a later date when the user may have developed immunity via the presence of the correct ratio of IgG/IgM antibodies or only IgG antibodies.

In another embodiment, a user may link to the analytical toilet system to substantiate their identity, leave a sample of excreta or sputum to be tested, then have the option of keeping the information private or sending it directly to an authority for post-infection clearance. The results may or not be permanently linked to the user.

In preferred embodiments, the antibody information collected from the analytical toilet is sent directly to a governmental authority, athletic competition authority, transportation-based company, or other authority such as those previously listed herein. In some instances, the authority only receives indication of pass or fail without receiving additional health and wellness information that may have been collected by the analytical toilet. This provides enough information to an authority to give or not to give post-infection clearance to a user but protects the information of the user and keeps it confidential to the user and maybe their health care provider. At any time, the clearance of a user may be revoked by one or more authorities. This may be done to remove one or more people from a venue such as a governmental building.

When the user uses the analytical toilet, a time stamp may also be associated with the antibody analysis information collected by the toilet. This is especially important for control of the spreading of contagious diseases amongst the general public. For example, if it is determined that antibodies for a specific contagious disease, such as a strain of coronavirus, only resides in a human subject for period of about one year, then the authority may require the user to be tested for antibodies at least once every 365 days. Thus, the length of time between uses of the toilet for antibody testing may be disease dependent. The length of time required between using the analytical toilet may vary from country to country, region to region or if there is a threat of an epidemic or pandemic. In some instances, a transportation authority may require a test within a short time frame before boarding such as one or two hours prior. In any of the methods described to transfer antibody analysis data from an analytical toilet, the data may be uploaded to a cloud storage center. The data may be retrieved from the cloud by the user, an authority, or a health care provider.

In one embodiment of a system for obtaining clearance for a user comprises an analytical toilet further comprising a mechanism to identify the user and generate an identification signal, a bowl to receive excreta and sputum from the user, an analyzer configured to analyze the sample to detect one or more antibodies in a sample of the excreta or sputum from the user and to generate an analysis signal, a processor configured to receive the identification signal and the analysis signal, determine therefrom the status of the user with respect to the one or more antibodies, and transmit a report to an authority, the report comprising the identity of the user and the status of the user with respect to the antibodies detected

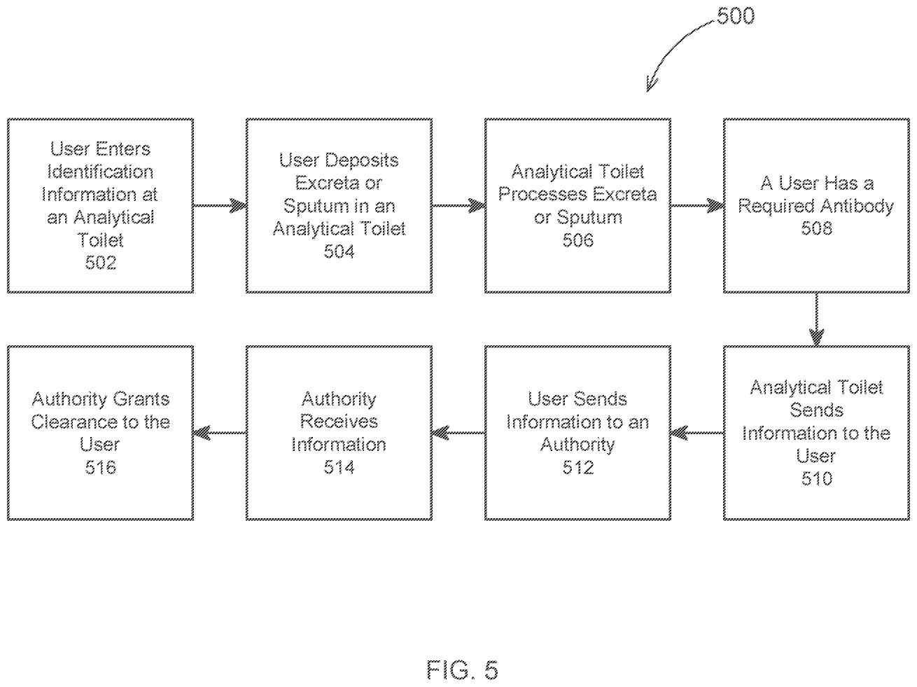

FIG. 5 illustrates the various steps of a method 500 to obtain post-infection clearance, according to an embodiment of the disclosure. The method comprises:

1. User Enters Identification Information at an Analytical Toilet 502;

2. User Deposits Excreta or Sputum in an Analytical Toilet 504;

3. Analytical Toilet Processes Excreta or Sputum 506;

4. A User Has a Required Antibody 508;

5. Analytical Toilet Sends Information to the User 510;

6. User Sends Information to an Authority 512;

7. Authority Receives Information 514; and

8. Authority Grants Clearance to the User 516.

Following use of the analytical toilet, the toilet may prepare the system for future analysis by removing from the test area waste products and other things that might contaminate the next analysis. This could include flushing the detection system, adding a buffer or stabilizing solution, or adding a gas to remove all liquid from the system. There are various options to clean, sanitize, and/or prepare the various components of the detection system between uses of the toilet. In one preferred embodiment, hot water is run through the fluidic circuits. In another preferred embodiment, oxygenated water is run through the fluidic circuits. In yet another preferred embodiment, a gas is run through the fluidic circuits to remove any liquid from being in contact with the detection system. Alternatively, cleaning and/or preservation agents, such as a detergent, are run through the fluid circuits.

In various exemplary embodiments, the lid may contain health and wellness sensors that interact with the user's back or that analyze gases in the bowl after the lid is closed.

In various exemplary embodiments, the analytical toilet includes software and hardware controls that are pre-set so that any manufacturer can configure their devices (i.e., analytical test devices) to work in the system. In a preferred embodiment, the system includes a software stack that allows for data channels to transfer data from the sensors in the medical toilet to cloud data systems. The software and hardware controls and/or software stack may be stored in the analytical toilet or remotely. This would allow scientists to place sensors, reagents, etc. in the system to obtain data for their research. It also allows user data to be individually processed, analyzed, and delivered to the user, or their health care provider, digitally (e.g., on a phone, tablet, or computer application). The seat may also contain sensors to measure fluid levels in the toilet. This could include proximity sensors. Alternatively, tubes in fluid communication with the bowl water could be used to determine changes to bowl fluids (e.g., volume, temperature, rate of changes, etc.).

The toilet disclosed herein has many possible uses, including private and public use. Whether for use by one individual, a small group of known users, or general public use, the toilet can detect, monitor, and create one-time and/or trend data for a variety of antibodies. This data can be used to prompt a user to seek additional medical, health, or wellness advice or treatment; track or monitor a user or population's immunity levels; and provide early detection or anticipation of a disease or another condition of which a user or population may wish to be aware. The analytical toilet described herein may be used for travelers seeking to pass through a continental, national, state, city, county, region or other border. Mobile units may be developed for a population under quarantine.

While the present disclosure often notes the detection system and other components supporting excreta and sputum analysis are located within the toilet, it is possible that some or all of the components are located outside of the toilet. For example, the sample preparation, detection, and processing equipment may be a separate unit adjacent to the toilet which cooperates with the toilet to automatically or semi-automatically receive excreta or sputum, prepare a sample of excreta or sputum for analysis, test the sample, discard the sample, and prevent cross contamination by cleaning and/or sterilizing portions of the toilet and external equipment that do any portion of the described process.

### EXAMPLES

The following examples are provided as part of the disclosure as an embodiment of the present invention. As such, none of the information provided below is to be taken as limiting the scope of the invention.

### Example 1. Method of Obtaining Travel Clearance

Example 1 is illustrative of a preferred method of obtaining travel clearance. The method comprises:
1. A user downloads an application on to their mobile phone. The user sets up an account over an encrypted network and enters driver's license and social security information.
2. A user electronically connects to the analytical toilet by exposing the QR code from the application on the user's phone or tablet to the user interface of the analytical toilet.
3. A user deposits a sample of sputum into the analytical toilet.
4. The analytical toilet transports the sample of sputum through a passage to the manifold. The manifold prepares the sample and delivers it to the detection system.
5. The detection system does not detect the presence of a contagious virus material.
6. The detection system relays the computer-readable data to a processor.
7. The processor processes the data and relays the information to the application on the user's mobile phone.
8. The user relays the information to the U.S. Transportation Safety Administration.
9. The U.S. Transportation Safety Administration grants the user clearance to travel aboard an airline for the next 365 days.
10. The clearance to travel information appears on the boarding pass of the user.
11. The analytical toilet flushes and cleans the detection system and bowl in preparation for the next user.

### Example 2. Method of Testing for SARS-CoV2-2019 Antibodies

Example 2 is illustrative of a preferred method of testing for SARS-CoV-2019 antibodies. The method comprises:
1. A user downloads an application on to their mobile phone. The user sets up an account over an encrypted network and enters driver's license and social security information.
2. A user electronically connects to the analytical toilet by exposing the QR code from the application on the user's phone or tablet to the user interface of the analytical toilet.
3. A user deposits a sample of sputum into the analytical toilet.
4. The analytical toilet transports the sample of sputum through a passage to the manifold. The manifold prepares the sample and delivers it to the detection system.
5. A 10 μL sample is deposited into center of the specimen well (S well) of a Cellex qSARS-CoV-2 IgG/IgM Rapid Test cassette at a temperature range of 2-30° C.
6. Two drops of sample diluent composed of 0.01M phosphate-buffered saline (PBS) solution of pH=7.4 is then dispensed immediately into the S well.
7. The sample and sample diluent are allowed to pass through the cassette for 20 min.
8. A sensor in the analytical toilet monitors the emergence of a burgundy colored stripe at the C line (control), G line (IgG) or M line (IgM).
9. The sensor detects a distinct burgundy stripe at the C line and G line indicating that the user comprises IgG antibodies only.
10. The detection system relays the computer-readable data to a processor.
11. The processor processes the data and relays the information to the application on the user's mobile phone.
12. The user relays the information to the U.S. Department of Homeland Security.
13. The U.S. Department of Homeland Security grants the user clearance to return to the workforce for the next 365 days.
14. The analytical toilet flushes and cleans the detection system and bowl and replaces the Cellex qSARS-CoV-2 IgG/IgM Rapid Test cassette in preparation for the next user.
15. The Cellex qSARS-CoV-2 IgG/IgM Rapid Test cassette is ejected and placed in a biohazard container for disposal.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. A system for obtaining clearance for a user comprising:
a mechanism to identify the user and generate an identification signal;
an analytical toilet comprising:
a bowl to receive excreta or sputum from the user;
an analyzer configured to analyze the sample to detect one or more antibodies in a sample of the excreta or sputum and to generate an analysis signal;
a processor configured to:
receive the identification signal and the analysis signal;
determine therefrom the status of the user with respect to the one or more antibodies; and
transmit a report to an authority, the report comprising the identity of the user and the status of the user with respect to the antibodies detected.

2. The system of claim 1 wherein the analyzer comprises:
an analysis chamber
a sample mechanism to add the sample to the analysis chamber;
a reagent mechanism to add an immunoagglutination reagent to the chamber;
a sensor to detect agglutination in the analysis chamber, indicating the presence of the one or more antibodies in the sample; and
a flush mechanism to wash the analysis chamber after each analysis.

3. The system of claim 1 wherein the analyzer comprises:
a plurality of cartridges, each configured to run a one-time immunoassay; and
an aligning mechanism configured to align a single cartridge with the sample to be analyzed for each excreta event.

4. The system of claim 3, wherein the aligning mechanism is configured to move the single cartridge into alignment with a port through which the sample enters the cartridge and into alignment with a sensor to detect the result of the one-time immunoassay.

5. The system of claim 3 wherein the one-time immunoassay is an enzyme-linked immunosorbent assay.

6. The system of claim 3 wherein the one-time immunoassay is a lateral flow assay.

7. The system of claim 1 wherein the report is transmitted to the authority by printing an optical readable code on a tangible medium or a digitally displayable image.

8. The system of claim 1 wherein the analyzer is configured to measure both IgG antibodies and IgM antibodies and the processor is configured to determine the ratio between the measured IgG antibodies and the measured IgM antibodies.

9. A method for obtaining travel clearance for a user comprising:
linking a user to an analytical toilet;
receiving a sample of excreta or sputum from the user in the analytical toilet;
analyzing the sample of excreta or sputum in an analytical toilet to detect one or more antibodies;
processing results from the analytical toilet and determining the status of the user with respect to the antibodies detected; and
submitting the identity of the user and the status of the user with respect to the antibodies detected to an authority for the purpose of gaining clearance from the authority.

10. The method of claim 9 wherein the user links to the analytical toilet by logging in wirelessly or on an interface, manually entering identifiable information on an interface, biometric scan, passport scan, driver's license scan, by a Bluetooth connection with a smart device, or by reading a QR code unique to the user.

11. The method of claim 9 wherein the antibodies detected are caused by infection of a disease selected from the group consisting of alphacoronavirus, betacoronavirus, gammacoronavirus, deltacoronavirus, SARS-CoV, SARS-CoV-2, MERS-CoV, influenza A, influenza B, influenza C, Ebola, dengue, viral meningitis, Zika, hepatitis A-E, tuberculosis, tetanus, typhoid fever, typhus, leprosy, diphtheria, streptococcus, staphylococcus, pertussis, chlamydia, bacterial meningitis, mycoplasma pneumonia, and any variants thereof.

12. A method for obtaining clearance for a user comprising:
providing an analytical toilet system with the capability to identify the user and to detect one or more antibodies by analyzing a sample of excreta or sputum from the user;
identifying the user;
receiving a sample of excreta or sputum from the user in the analytical toilet;
analyzing the sample in the analytical toilet to detect the one or more antibodies;
determining the status of the user with respect to the one or more antibodies based on the result of the analysis; and
submitting to an authority the identity of the user and the status of the user with respect to the antibodies detected for the purpose of obtaining clearance from the authority.

13. The method of claim 12, wherein the one or more antibodies detected are from an infection by a bacteria or virus.

14. The method of claim 13 wherein the virus is selected from the group consisting of alphacoronavirus, betacoronavirus, gammacoronavirus, deltacoronavirus, SARS-CoV, SARS-CoV-2, MERS-CoV, influenza A, influenza B, influenza C, Ebola, dengue, viral meningitis, hepatitis A-E, Zika, and any variants thereof.

15. The method of claim 13 wherein the bacteria is selected from the group consisting of tuberculosis, tetanus, typhoid fever, typhus, leprosy, diphtheria, streptococcus, staphylococcus, pertussis, chlamydia, bacterial meningitis, mycoplasma pneumonia, and any variants thereof.

16. The method of claim 12 wherein the analyzing step involves an immunoassay.

17. The method of claim 16 wherein the immunoassay uses a mechanism selected from the group consisting of immunoagglutination, immunosorbent, and immunoblotting.

18. The method of claim 12 wherein analyzing the sample comprises a western blot test, lateral flow immunoassay (LFA) or an enzyme-linked immunosorbent assay (ELISA).

19. The method of claim 18 wherein the western blot test, lateral flow immunoassay (LFA) or an enzyme-linked immunosorbent assay (ELISA) further comprises a recombinant protein.

20. The method of claim 19 wherein the recombinant protein is a portion of a bacteria or virus.
